(19) United States
(12) Patent Application Publication
(10) Pub. No.: US 2024/0207261 A1
(43) Pub. Date: Jun. 27, 2024

(54) METHODS FOR TREATING SMALL CELL LUNG CANCER AND OTHER NEUROENDOCRINE CANCERS

(71) Applicant: BOARD OF REGENTS, THE UNIVERSITY OF TEXAS SYSTEM, Austin, TX (US)

(72) Inventors: Lauren A. BYERS, Houston, TX (US); Carl M. GAY, Houston, TX (US)

(73) Assignee: BOARD OF REGENTS, THE UNIVERSITY OF TEXAS SYSTEM, Austin, TX (US)

(21) Appl. No.: 18/549,857

(22) PCT Filed: Mar. 10, 2022
(86) PCT No.: PCT/US2022/019756

§ 371 Date: Sep. 8, 2023

Related U.S. Application Data
(60) Provisional application No. 63/159,401, filed on Mar. 10, 2021.

(51) Int. Cl. A61K 31/4995; A61P 35/00; G01N 33/574

(52) U.S. Cl. CPC A61K 31/4995; A61P 35/00; G01N 33/57423

(57) ABSTRACT

The current disclosure provides novel therapeutic methods for treating SCLC and other neuroendocrine cancers by evaluating the biomarker SLFN 11. Aspects of the disclosure relate to a method for treating a subject with small cell lung cancer (SCLC) or with a neuroendocrine cancer, the method comprising administering one or more therapeutics to a subject that has had been evaluated for SLFN 11 expression in a biological sample from the subject; wherein the one or more therapeutics comprise lurbinectedin.

This application is claims benefit of priority of U.S. Provisional Application No. 63/159,401, filed Mar. 10, 2021, which is hereby incorporated by reference in its entirety.

### FIELD OF THE INVENTION

The present invention relates generally to the fields of molecular biology and therapeutic diagnosis. More particularly, it concerns methods and compositions involving prognosing, diagnosing, and treating SCLC and other neuroendocrine cancers.

### I. BACKGROUND

Small cell lung cancer (SCLC) is an aggressive subtype of lung cancer, accounting for approximately 15% of all lung cancer cases in United States. SCLC is characterized by small cells with poorly defined cell borders and minimal cytoplasm, rare nucleoli, and finely granular chromatin. Due to the aggressive nature of the disease, the low rate of early diagnosis, and the lack of effective therapies, prognosis is generally poor. Median survival time from diagnosis for untreated SCLC patients is only two to four months. When chemotherapy and/or radiation modalities are used, the initial response rate to among SCLC patients is high (approximately 60 to 80%), but relapse occurs in the majority treated patients, who then are largely refractory to further systemic therapy. Thus, even with current treatment modalities, the median survival time for patients with limited-stage disease is 16 to 24 months and for patients with extensive disease, seven to 12 months. To improve patient survival rates, it is essential to treat patients with chemotherapeutic agents to which their tumors are sensitive. Use of targeted drugs in the treatment of SCLC represents a major unmet medical need. Unlike non-small cell lung cancers (NSCLC), there are currently no targeted therapies with demonstrated benefit for patients with this disease. Thus, there is a need to align SCLC patients with suitable treatments based on their individual genetic profiles. Understanding a given tumor's genetic profile will also enable early diagnosis, detection, and treatment selection.

### SUMMARY

The current disclosure provides novel therapeutic methods for treating SCLC and other neuroendocrine cancers by evaluating the biomarker SLFN11. Aspects of the disclosure relate to a method for treating a subject with small cell lung cancer (SCLC) or with a neuroendocrine cancer, the method comprising administering one or more therapeutics to a subject that has had been evaluated for SLFN11 expression in a biological sample from the subject; wherein the one or more therapeutics comprise lurbinectedin. Further aspects relate to a method for predicting a response to lurbinectedin in a subject having a neuroendocrine cancer or SCLC comprising a) evaluating SLFN11 in a biological sample from the subject; b) predicting that the subject will respond to lurbinectedin after (i) SLFN11 expression is detected in the biological sample from the patient; (ii) the patient is determined to have high expression of SLFN11 compared to a control wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04; or (iii) the H-score for the level of expression in the biological sample from the subject is greater than or equal to 1; or c) predicting that the subject will not respond to lurbinectedin after (i) SLFN11 expression is not detected in the biological sample from the patient; (ii) the patient is determined to have low or substantially the same level of SLFN11 expression compared to a control wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04; or (iii) the H-score for the level of expression in the biological sample from the subject is less than 1. Yet further aspects relate to a method for evaluating a subject comprising detecting SLFN11 in a biological sample from the subject.

The cancer may be a SCLC or neuroendocrine cancer or the subject may be one that has been diagnosed or determined to have SCLC or a neuroendocrine cancer. In some aspects, the subject was evaluated for, or the methods comprise evaluating or determining SLFN11 expression by evaluating SLFN11 in an immunohistochemistry assay performed on a biological sample from the subject. In some aspects, the subject was evaluated for SLFN11 expression by detecting binding of the SLFN11 protein to an anti-SLFN11 antibody, wherein the anti-SLFN11 antibody comprises clone D8W1B. In some aspects, the subject was evaluated for SLFN11 expression by detecting binding of the SLFN11 protein to an anti-SLFN11 antibody, wherein the anti-SLFN11 antibody comprises HPA023030. HPA023030 is available commercially (Sigma-Aldrich Cat #HPA023030, RRID:AB_1856613). In some aspects, the biological sample comprises a liquid biopsy, pleural effusion, blood, serum, plasma, biopsy, or tissue sample. The biological sample may also be a biological sample described herein. In some aspects, the biological sample comprises circulating tumor cells. In some aspects, the expression level of SLFN11 in the biological sample from the subject has been quantitated or the method further comprises quantitating the expression level of SLFN11. In some aspects, the expression level of SLFN11 is normalized or the method further comprises normalizing the expression level of SLFN11. In some aspects, the subject has been determined to be positive for SLFN11 expression in the biological sample. In some aspects, the subject has been determined to have SLFN11+ cells in the biological sample from the subject. In some aspects, the subject has or has been determined to have a high level of SLFN11 expression in the biological sample from the subject. In some aspects, the subject has or has been determined to have a high level of SLFN11 expression in the biological sample from the subject compared to a control level of expression. In some aspect, the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04. In some aspects, high expression is defined as at least one standard deviation higher than a control. In some aspects, the therapeutic excludes an ATR inhibitor. In some aspects, the therapeutic excludes berzosertib. In some aspects, the control comprises a cut-off value above which, high expression of SLFN11 is defined and below which, low expression of SLFN11 is defined. In some aspects, the cut-off value is further defined as an H-score, which is defined as the percentage of cells expressing SLFN11 times the intensity of SLFN11 staining (0 [meaning no staining/expression] vs 1+/2+/3+ for a range of possible H-scores of 0-300). In some aspects, the H-score is 1. In some aspects, the H-score is at least, H-score is at most, or H-score is exactly 0.1, 0.5, 0.8, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any derivable range therein. Further aspects are described in Allison Stewart C, et al., Oncotarget. 2017 Apr. 25; 8(17):28575-28587 and Pietanza M C, et al., LA. J Clin Oncol. 2018 Aug. 10; 36(23):2386-2394, which are herein incorporated by reference. In some aspects, the methods further comprise determining the H-score for SLFN11 expression in the subject. In some aspects, the subject is determined to have a H-score that is greater than 1. In some aspects, the subject is determined to have a H-score that is less than 1. In some aspects, the subject is determined to have a H-score of 1 or greater. In some aspects, the subject is determined to have a H-score of 1 or less. In some aspects, the subject is determined to have a H-score of greater than or equal to 1.

In some aspects, the subject has not previously been treated with lurbinectedin. In some aspects, the subject has been previously treated with a cancer therapeutic. In some aspects, the subject has been previously treated with a platinum-based chemotherapeutic. In some aspects, the subject has not been previously treated with a platinum-based chemotherapy. The subject may also be further defined as currently taking a platinum-based chemoterhapy or one who has relapsed after platinum-based chemotherapeutic treatment. The platinum-based chemotherapeutic may include cisplatin, oxaliplatin, carboplatin, and combinations thereof. In some aspects, the cancer is further defined as recurrent. In some aspects, the cancer comprises SCLC type A. The subject may be a human, mammalian, non-human primate, rat, mouse, pig, horse, cat, or dog. In some aspects, the subject is a human.

In some aspects, the expression level of the measured biomarkers are/were determined to be significantly different as compared to a control level of expression, wherein the control level of expression comprises the level of expression of the biomarkers in a cancer that is not a SCLC or neuroendocrine cancer. In some aspects, the expression level of the measured biomarkers are/were determined to be not significantly different as compared to a control level of expression, wherein the control level of expression comprises the level of expression of the biomarkers in a non-cancerous sample. In some aspects, the expression level of the measured biomarkers are/were determined to be significantly different as compared to a control level of expression, wherein the control level of expression comprises the level of expression of the biomarkers in a non-cancerous sample. In some aspects, the expression level of the measured biomarkers are/were determined to be not significantly different as compared to a control level of expression, wherein the control level of expression comprises the level of expression of the biomarkers in a cancerous sample, wherein the cancer comprises a cancer described herein. In some aspects, the expression level of the measured biomarkers are/were determined to be significantly different as compared to a control level of expression, wherein the control level of expression comprises the level of expression of the biomarkers in a cancerous sample, wherein the cancer comprises a cancer described herein.

In some aspects, the one or more biomarkers has an absolute value of the signature weight of greater than 0.025. The term “absolute value” refers to the magnitude of a real number without regard to its sign. Signature weights are listed in Tables 1-3. In some aspects, the one or more biomarkers has an absolute value of the signature weight of greater than or less than 0.000001, 0.000002, 0.000003, 0.000004, 0.000005, 0.000006, 0.000007, 0.000008, 0.000009, 0.00001, 0.000015, 0.00002, 0.000025, 0.00003, 0.000035, 0.00004, 0.000045, 0.00005, 0.000055, 0.00006, 0.000065, 0.00007, 0.000075, 0.00008, 0.000085, 0.00009, 0.000095, 0.0001, 0.00015, 0.0002, 0.00025, 0.0003, 0.00035, 0.0004, 0.00045, 0.0005, 0.00055, 0.0006, 0.00065, 0.0007, 0.00075, 0.0008, 0.00085, 0.0009, 0.00095, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, or 0.05 (or any range derivable therein).

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect or embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is specifically contemplated that any limitation discussed with respect to one embodiment or aspect of the invention may apply to any other embodiment and/or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments and aspects discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows results from analysis of lurbinected in activity on 12 human-derived SCLC cell lines.

FIG. 2A-2B. FIG. 2A shows a comparison of SCLC subtype-specific lurbinectedin IC₅₀ values demonstrating no significant differences among SCLC-A, N, P and I. FIG. 2B shows a comparison of lurbinectedin IC₅₀ values between high SLFN11 expressing cells (High) and low SLFN11 expressing cells (Low).

FIG. 3 shows results from an analysis of biomarkers of response to lurbinectedin, determined by comparing RPPA marker expression from the most sensitive cell lines to less sensitive cell lines (fold change, p-value by t-test).

FIG. 4A-4C shows Western Blot analysis results demonstrating that DNA damage markers are upregulated in SCLC cells upon treatment with lurbinectedin.

FIG. 5A-5C shows that cell lines with high protein expression of SLFN11 (SCLC cells) showed significantly greater sensitivity to Lurbinectedin (p=0.004, by t-test) based on differences in their IC50 values (lower IC50=more sensitive) (A-B). C demonstrates induction of DNA damage with lurbinectedin treatment.

FIG. 6A-6H. SLFN11 predicts sensitivity to lurbinectedin in SCLC. A. Sensitivity of a panel of 21 human SCLC cell lines to lurbinectedin (ranked by IC₅₀) and color coded for SCLC subtypes. B. Spearman correlation of RPPA protein markers and IC₅₀ values in panel of 21 cell lines (p<0.05). C. Comparison of lurbinectedin IC₅₀ values between cell lines with high and low SLFN11 expression by RPPA (bi-modal separation, mean±SEM, p-value by t-test). D. Comparison of lurbinectedin IC₅₀ values between cell lines from the four SCLC subtypes SCLA-A (n=12), -N(n=4), -P (n=3) and -I (n=2) (p-values by t-test). E. Western-blot showing knockdown of SLFN11 following siRNA DMS79 and H209 cell lines. F. Sensitivity of cell lines following SLFN11 knockdown. G, H. Tumor growth curves of SLFN11 high xenografts (DMS79) and SLFN11 low xenografts (H865) treated with vehicle, lurbinectedin (0.1 mg/kg), or cisplatin (6 mg/kg) (** p=0.01 by un-paired t-test).

FIG. 7A-7B. Induction of DNA damage and PD-Li expression following lurbinectedin treatment. A. Western-blot showing changes in replication stress markers (pCHK1 and pRPA32) and DNA damage (pγH2Ax) in SCLC cell lines following 24- and 48-hours treatment with DMSO or lurbinectedin (Lurbi, 0.9 nM). B. Treatment with lurbinectedin activates cGAS, phosphorylates STING and increases PD-L1 expression in SCLC-P and SCLC-I cell lines.

FIG. 8A-8B. SLFN11 predicts sensitivity to lurbinectedin in SCLC-A cell lines. A. Spearman correlation of RPPA protein markers and IC50 values in panel of 12 SCLC-A cell lines (FDR<0.2). B. Comparison of lurbinectedin IC50 values between SCLC-A cell lines with high and low SLFN11 expression by RPPA (bi-modal separation, mean±SEM, p-value by student's t-test).

FIG. 9A-9C. A. Western-blot showing changes in replication stress markers (pCHK1 and pRPA32) and DNA damage e(γH2Ax) in SCLC cell lines following 24- and 48-hours treatment with DMSO or lurbinectedin (Lurbi,0.9 nM). B. Treatment with lurbinected inactivates cGAS, phosphorylates STING and increases PD-L1 expression in SCLC-P and SCLC-I cell lines. C. Western blots showing changes in SLFN11 expression following lurbinectedin treatment.

### DETAILED DESCRIPTION OF THE INVENTION

### I. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from lung tissue by any of the biopsy methods previously mentioned. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen. In some aspects, the sample includes pleural effusions or blood.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects, the sample comprises a fractionated sample, such as a blood sample that has been fractionated by centrifugation or other fractionation technique. The sample may be enriched in white blood cells or red blood cells. In some aspects, the sample may be fractionated or enriched for leukocytes or lymphocytes. In some aspects, the sample comprises a whole blood sample. In some aspects, the sample comprises circulating tumor cells. In some aspects, the sample comprise circulating tumor DNA.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a esophageal or a suspected esophageal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### II. Cancer Monitoring

In certain aspects, the methods of the disclosure may be combined with one or more other cancer diagnosis or screening tests at increased frequency if the patient is determined to be at high risk for recurrence or have a poor prognosis based on the biomarker expression described above.

In some aspects, the methods of the disclosure further include one or more monitoring tests. The monitoring protocol may include any methods known in the art. In particular, the monitoring include obtaining a sample and testing the sample for diagnosis. For example, the monitoring may include endoscopy, biopsy, endoscopic ultrasound, X-ray, barium swallow, a Ct scan, a MRI, a PET scan, laparoscopy, or cancer biomarker testing. In some aspects, the monitoring test comprises radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

Methods of the disclosure may further include one or more of a urinalysis, urine cytology, urine culture, or urine tumor marker tests. Different urine tests look for specific substances made by cancer cells. One or more of these tests may be used in the methods of the disclosure. These include the tests called NMP22® (or BladderChek®), BTA Stat®, Immunocyt®, and UroVysion®. Methods of the disclosure also include cystoscopy. In this method, a urologist uses a cystoscope, which is a long, thin, flexible tube with a light and a lens or a small video camera on the end. Fluorescence cystoscopy (also known as blue light cystoscopy) may be done along with routine cystoscopy. For this exam, a light-activated drug is put into the bladder during cystoscopy. It's taken up by cancer cells. When the doctor then shines a blue light through the cystoscope, any cells containing the drug will glow (fluoresce). This can help the doctor see abnormal areas that might have been missed by the white light normally used.

Methods of the disclosure also include the use of transurethral resection of bladder tumor (TURBT). The procedure used to biopsy an abnormal area is a transurethral resection of bladder tumor (TURBT), also known as just a transurethral resection (TUR). During this procedure, the doctor removes the tumor and some of the bladder muscle around the tumor. The removed samples are then sent to a lab to look for cancer. Bladder cancer can sometimes start in more than one area of the bladder (or in other parts of the urinary tract). Because of this, the doctor may take samples from many different parts of the bladder, especially if cancer is strongly suspected but no tumor can be seen. Salt water washings of the inside the bladder may also be collected and tested for cancer cells.

In some aspects, imaging tests are performed or the subject is one that has undergone an imaging test. Imaging tests may use x-rays, magnetic fields, sound waves, or radioactive substances. In some aspects, the imaging test comprises an Intravenous pyelogram (IVP). An intravenous pyelogram (IVP), also called an intravenous urogram (IVU), is an x-ray of all of the urinary system taken after injecting a special dye into a vein. This dye is removed from the bloodstream by the kidneys and then passes into the ureters and bladder. X-rays are done while this is happening. The dye outlines these organs on the x-rays and helps show urinary tract tumors. In some aspects, the imaging test comprises a retrograde pyelogram. For this test, a catheter (thin tube) is put in through the urethra and up into the bladder or into a ureter. Then a dye is injected through the catheter to make the lining of the bladder, ureters, and kidneys easier to see on x-rays. In some aspects, the imaging test comprises computed tomography (CT) scan. A CT scan uses x-rays to make detailed cross-sectional pictures of the body. CT-guided needle biopsy: CT scans can also be used to guide a biopsy needle into a suspected tumor. This can be used to take samples from areas where the cancer may have spread. In some aspects, the imaging test comprises magnetic resonance imaging (MRI) scan. Like CT scans, MRI scans show detailed images of soft tissues in the body. But MRI scans use radio waves and strong magnets instead of x-rays. In some aspects, the imaging test comprises an ultrasound. Ultrasound uses sound waves to create pictures of internal organs. Ultrasound can also be used to guide a biopsy needle into a suspected area of cancer in the abdomen or pelvis. In some aspects, the imaging test comprises a chest x-ray or bone scan. A chest x-ray or bone scan may be done to see if the bladder cancer has spread to the lungs or bone, respectively.

### III. ROC Analysis

In statistics, a receiver operating characteristic (ROC), or ROC curve, is a graphical plot that illustrates the performance of a binary classifier system as its discrimination threshold is varied. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1−specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### IV. Nucleic Acid Assays

Aspects of the methods include assaying nucleic acids to determine expression or activity levels and/or the presence of CXCL13 expressing cells and/or ARID1A mutant cells in a biological sample. Arrays can be used to detect differences between two samples. Specifically contemplated applications include identifying and/or quantifying differences between RNA from a sample that is normal and from a sample that is not normal, between a cancerous condition and a non-cancerous condition. Also, RNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

To determine expression levels of a biomarker, an array may be used. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

Further assays useful for determining biomarker expression include, but are not limited to, nucleic amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

A further assay useful for quantifying and/or identifying nucleic acids, such as nucleic acids comprising biomarker genes, is RNAseq. RNA-seq (RNA sequencing), also called whole transcriptome shotgun sequencing, uses next-generation sequencing (NGS) to reveal the presence and quantity of RNA in a biological sample at a given moment in time. RNA-Seq is used to analyze the continually changing cellular transcriptome. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

### V. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, flow cytometry, or immunofluorescence techniques to detect biomarker expression such as CXCL13. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

### VI. Administration of Therapeutic Compositions

The therapy provided herein may comprise administration of a combination of therapeutic agents, such as a first anticancer therapy and a second anticancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. Methods of Treatment

Provided herein are methods for treating or delaying progression of cancer in an subject through the administration of therapeutic compositions.

In some aspects, the therapies result in a sustained response in the individual after cessation of the treatment. The methods described herein may find use in treating conditions where enhanced immunogenicity is desired such as increasing tumor immunogenicity for the treatment of cancer.

In some aspects, the individual has cancer that is resistant (has been demonstrated to be resistant) to one or more anticancer therapies. In some aspects, resistance to anticancer therapy includes recurrence of cancer or refractory cancer. Recurrence may refer to the reappearance of cancer, in the original site or a new site, after treatment. In some aspects, resistance to anticancer therapy includes progression of the cancer during treatment with the anticancer therapy. In some aspects, the cancer is at early stage or at late stage.

In some aspects of the methods of the present disclosure, the cancer has low levels of T cell infiltration. In some aspects, the cancer has no detectable T cell infiltrate. In some aspects, the cancer is a non-immunogenic cancer (e.g., non-immunogenic colorectal cancer and/or ovarian cancer). Without being bound by theory, the combination treatment may increase T cell (e.g., CD4+ T cell, CD8+ T cell, memory T cell) priming, activation, proliferation, and/or infiltration relative to prior to the administration of the combination.

The cancer may be a solid tumor, metastatic cancer, or non-metastatic cancer. In certain aspects, the cancer may originate in neuroendocrine cells.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

In certain aspects, further cancer or metastasis examination or screening, or further diagnosis such as contrast enhanced computed tomography (CT), positron emission tomography-CT (PET-CT), and magnetic resonance imaging (MRI) may be performed for the detection of cancer or cancer metastasis in patients determined to have a certain gut microbiome composition.

Methods of the disclosure relate to the treatment of subjects with cancer. In some aspects, the methods may be employed with respect to individuals who have tested positive for such cancer, who have one or more symptoms of a cancer, or who are deemed to be at risk for developing such a cancer.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### IX. Example 1—Characterization of Lurbinectedin as a Single Agent and in Combinations with DNA Damage Response Inhibitors for the Treatment and Bio-Marker Discovery of SCLC

Small-cell lung cancer (SCLC) is an aggressive neuroendocrine lung malignancy which is primarily driven by loss of function of tumor suppressor gene TP53 and RB1 and accounts for around 13-15% of all lung cancers. For metastatic SCLC the standard-of-care, first-line therapy is combination of platinum-based therapy with etoposide or with irinotecan. Though response rate in first-line chemotherapy in SCLC is very high, relapse is almost universal. For decades topotecan, a topoisomerase-I inhibitor was the only FDA approved second-line treatment in SCLC. However, on Jun. 15, 2020 the FDA approved lurbinectedin for treatment of patients with metastatic SCLC with disease progression from platinum-based chemotherapy and designated it as an orphan drug. Lurbinectedin (PM01183) is a synthetic analog of the natural marine-based tetrahydroisoquinoline, trabectedin which comes from the sea-squirt species Ecteinascidia turbinate. Lurbinectedin blocks transcription by inhibiting the activity of RNA-polymerase-II and inducing its specific degradation by the ubiquitin/proteasome machinery, also inducing DNA damage. Emerging data supports that SCLC is transcriptionally addicted via one of three main transcription factors ASCL1 (A), NeuroD1 (N) & POU2F3 (P), which may contribute to the promising results observed in SCLC trials to date with lurbinectedin. However, there are currently no established biomarkers to predict SCLC sensitivity or resistance to lurbinectedin. Furthermore, little is known regarding molecular changes in SCLC cells or other tumors upon exposure to lurbinectedin. In this preclinical study the inventors investigated the therapeutic efficacy of lurbinectedin in large number of profiled SCLC cell lines representing the four SCLC subtypes (A, N, P and I) to identify candidate markers of drug sensitivity and resistance. In 12 human-derived and 3 mouse model-derived SCLC cell lines, the majority of cell lines were highly sensitive to lurbinectedin at a very low doses (median IC50 0.52 nM, range 0.08-1.84 nM). The inventors also observed increased markers of DNA damage following treatment in sensitive cell lines (e.g., TH2AX, ChK1, RPA32) by western blot. Notably, no subtype specific difference in lurbinectedin sensitivity was observed among the four subtypes (A, N, P and I) of SCLC cells. However, cell lines with lower SLFN11 expression were more sensitive to lurbinectidin. Western blot experiments showed significant increase in phosphorylation of TH2AX, ChK1, RPA32 in lurbinectedin treated SCLC cells compared to DMSO treatment. Together this data confirm lurbinectedin as a potent treatment option for SCLC, and support a rationale for potential combinations with other DNA damaging agents.

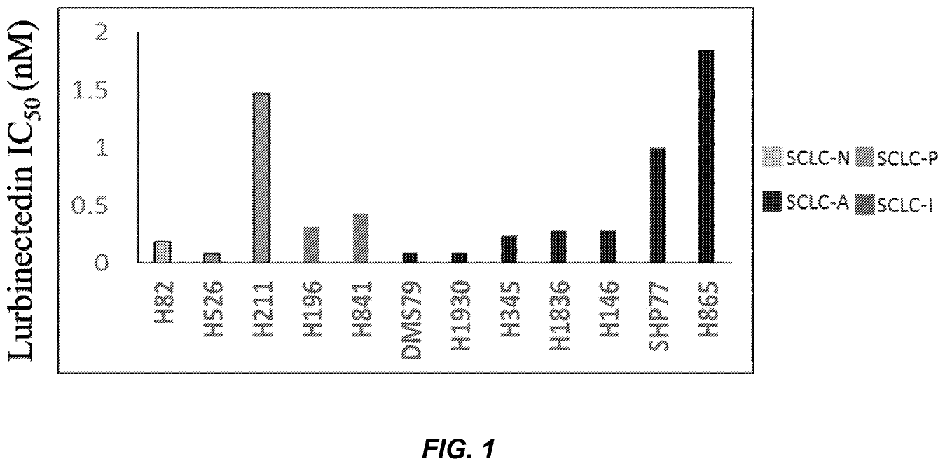

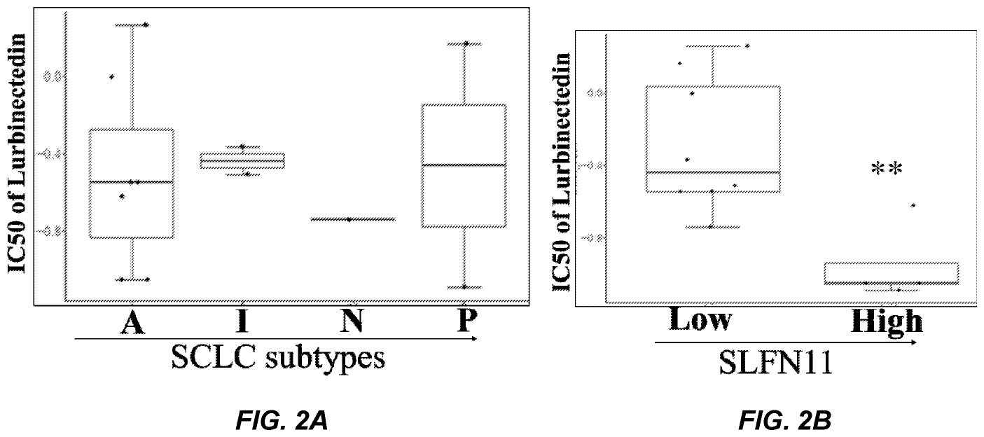

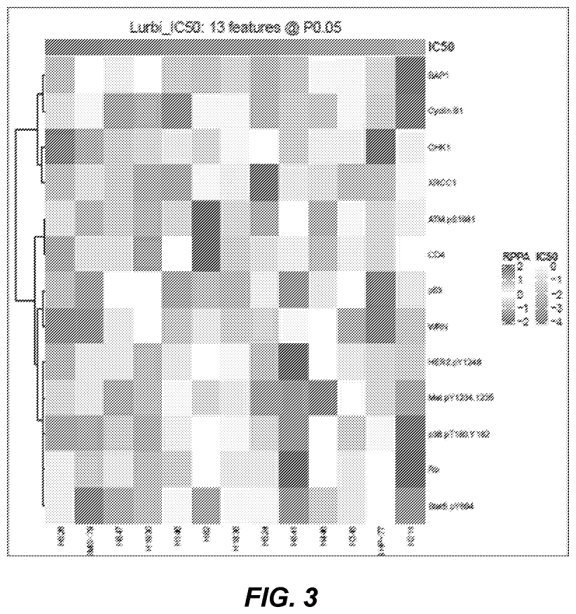

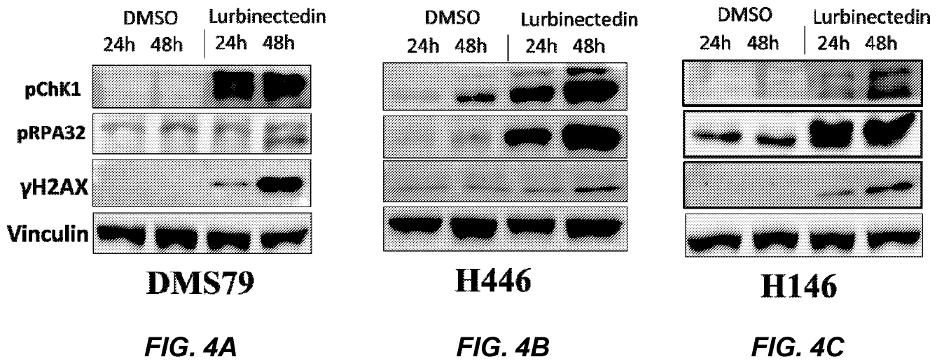

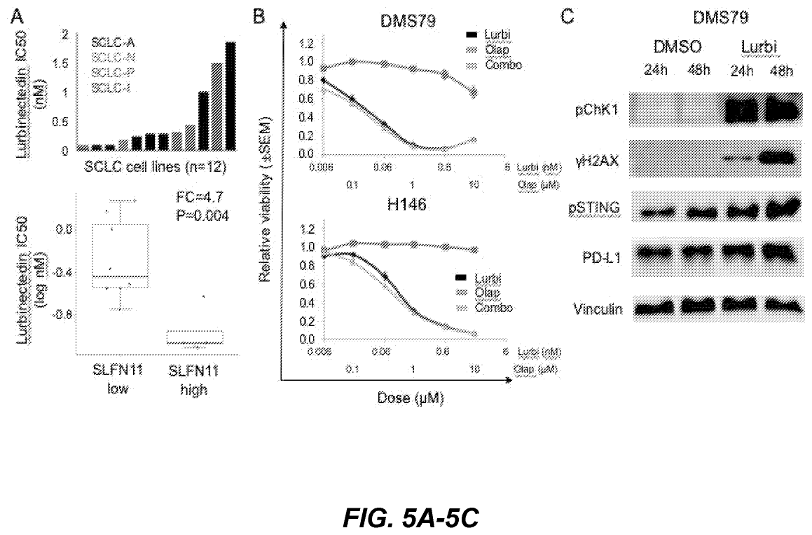

In this preclinical study, the inventors investigated the therapeutic efficacy of lurbinectedin in large number of profiled SCLC cell lines representing the four SCLC subtypes (A, N, P and I) to identify candidate markers of drug sensitivity and resistance. In 12 human-derived and 3 mouse model-derived SCLC cell lines, the majority of cell lines were highly sensitive to lurbinectedin at a very low doses (median IC₅₀ 0.52 nM, range 0.08-1.84 nM, FIG. 1). The inventors also observed increased markers of DNA damage following treatment with 1 nM lurbinectedin for 24 or 48 hours in sensitive cell lines (e.g., TH2AX, ChK1, RPA32) by western blot (FIGS. 4A-4C). Notably, no subtype specific difference in lurbinectedin sensitivity was observed among the four subtypes (A, N, P and I) of SCLC cells (FIG. 2A). However, cell lines with lower SLFN11 expression were more sensitive to lurbinectidin (FIG. 2B); SLFN11-high SCLC cells showed significantly (p=0.0035, by t-test) lower IC₅₀ than SLFN11-low cells. Western blot experiments showed significant increase in phosphorylation of TH2AX, ChK1, RPA32 in lurbinectedin treated SCLC cells compared to DMSO treatment. Together, these data confirm lurbinectedin as a potent treatment option for SCLC and support a rationale for potential combinations with other DNA damaging agents.

Methods: Drug Sensitivity:

A 12 SCLC cell line panel representing SCLC-A, N, P and I subtypes, developed at MD Anderson Cancer Center, was screened against lurbinectedin in 96 hr proliferation assays (FIG. 2). Western Blotting: Three SCLC cell lines were treated with 1 nM of lurbinectedin for 24 and 48h and then subjected for protein isolation and Western blotting for pChK1, pRPA32, TH2AX.

### Example 2: SLFN11 Biomarker Status Predicts Response to Lurbinectedin in Small Cell Lung Cancer

Lurbinectedin recently received FDA accelerated approval as a second line treatment option for metastatic small cell lung cancer (SCLC). However, there are currently no established biomarkers to predict SCLC sensitivity or resistance to lurbinectedin or preclinical studies to guide rational combinations.

Methods: Drug sensitivity was assayed in proliferation assays and xenograft models. Baseline proteomic profiling was performed by reverse-phase protein array. Lurbinectedin-induced changes in intracellular signaling pathways were assayed by Western blot.

Results: Among 21 human SCLC cell lines, cytotoxicity was observed following lurbinectedin treatment at a low doses (median IC50 0.46 nM, range 0.06-1.83 nM). Notably, cell lines with high expression of Schlafen-11 (SLFN11) protein were more sensitive to single-agent lurbinectedin (FC=3.2, p=0.005). SLFN11 was validated as a biomarker of sensitivity to lurbinectedin using siRNA knockdown and in xenografts representing SLFN11 high and low SCLC. Replication stress and DNA damage markers (e.g., TH2AX, phosphorylated CHK1, phosphorylated RPA32) increased in SCLC cell lines following treatment with lurbinectedin. Lurbinectedin also induced PD-L1 expression via cGAS-STING pathway activation

Conclusions: Together the data confirm the activity of lurbinectedin across a large cohort of SCLC models and identify SLFN11 as a top candidate biomarker for lurbinectedin sensitivity.

Small cell lung cancer (SCLC) is an aggressive form of high-grade neuroendocrine lung cancer with dismal patient outcome of a five-year survival of less than 10% (1). It is characterized by loss of function of the tumor suppressor genes TP53 and RB1, and accounts for around 13-15% of all lung cancers (1). For decades the first line treatment for extensive-stage SCLC, has been platinum-based chemotherapy combinations, with the incorporation of PD-L1 checkpoint blockade as part of the frontline regimen only since 2018 (2,3). In the second line setting, topotecan, a topoisomerase I inhibitor was the only US Food and Drug Administration (FDA) approved treatment (4) until the FDA recently granted accelerated approval to lurbinectedin for the treatment of patients with metastatic SCLC with disease progression on or after frontline platinum-based chemotherapy (4). In a single-arm, open-label, phase 2 basket trial overall response by investigator assessment was seen in 37 of 102 patients enrolled (35.2%; 95% CI 26.2-45.2) with an acceptable and manageable safety profile (5). Lurbinectedin (PM01183) is a synthetic analog of the natural marine-based tetrahydroisoquinoline, trabectedin which is derived from the sea squirt species Ecteinascidia turbinate (6,7). Lurbinectedin blocks the activity of RNA-polymerase-II and induces its degradation by the ubiquitin/proteasome machinery, also inducing DNA damage (8). By hampering the transcription processes within tumor-associated macrophages lurbinectedin also modifies tumor microenvironment (9). To date, no biomarkers of response to lurbinectedin have been reported in SCLC, or any other cancer type. Furthermore, little is known about how intra-cellular signaling is altered following treatment with lurbinectedin.

In this preclinical study, the inventors investigate the therapeutic efficacy of lurbinectedin in large panel of profiled cell lines representing the molecular heterogeneity of SCLC to identify candidate predictive biomarkers of response. They then characterized changes in DNA damage response pathway and immunogenic cell death associated pathway in SCLC cells upon treatment with lurbinectedin.

A. Materials & Methods:

1. Cell Lines and Cell Culture—

Human SCLC cell lines (21 cell lines) were purchased from ATCC. Cell lines were tested and authenticated by short tandem repeat profiling (DNA fingerprinting) and not cultured for greater than 6-months and were routinely tested for Mycoplasma contamination. Cell lines were cultured in either RPMI or HITES medium with 10% fetal bovine serum and 1% pen-strep, at 37° C. in a humidified chamber with 5% C02.

2. Chemical Compounds—

Lurbinectedin was purchased from MedChemExpress (NJ, USA) or acquired from the MD Anderson pharmacy. Compound(s) were dissolved in dimethyl sulfoxide (DMSO) for in vitro treatments.

3. Cell Proliferation Assay—

Cells were seeded in 96-well plates at 2,000 cells per well in triplicate for each cell line. After 24 hours, the cells in each well were treated with a lurbinectedin or with vehicle control. Four days later, proliferation was assayed by Cell Titer Glo (Promega, Fitchburg, WI). For single drug treatments, median inhibitory concentration (IC₅₀) values were estimated by the Drexplorer software using date from duplicate experiments (14). For drug combinations, the area under the curve (AUC) of the observed (or experimental) effect of the combination was compared to the predicted additive effect. Data was subsequently presented as a ratio of the experimental effect relative to the predicted additive effect based on the Bliss Independence model, as previously reported (15). Using 10% above or below the predicted additive effect as a cut-off, the inventors then assigned the following groups: Observed/predicted<−0.1=greater than additive; Observed/predicted <0.1=less than additive; Observed/predicted <0.1 and >−0.1=additive.

4. siRNA Based Knockdown of SLFN11—

For SLFN11 gene silencing, pooled small interfering RNAs (siRNAs) targeting SLFN11 (L-016764-01-0005) or its corresponding scramble control (D-001810-10-05; GE Dharmacon) were transfected into DMS79 and H209 cells for 72 hours using Lipofectamine 2000 (Invitrogen) according to the manufacturer's protocol. The cells were then plated for 96h proliferation assays. Knock down efficiency was validated by Western blotting.

5. Western Blot—

Protein lysate were isolated from SCLC cells treated with 0.9 nM lurbinectedin or DMSO for 24 and 48h. Nitrocellulose membranes were incubated with primary antibodies (1:1000) for Human SLFN11 (Santa Cruz Biotechnology), phospho-(S345) CHK1 Cell Signaling Technologies (CST), phospho-(S4/S8) RPA32 (Bethyl laboratories), phospho-(S139) TH2AX (CST), cleaved caspase-3 (CST), phospho-(S366) STING (SAB Biotech), cGAS (CST), PD-L1(CST), GAPDH (Santa Cruz Biotechnology) and Vinculin (Sigma-Aldrich) overnight. Secondary anti-goat, anti-mouse (Santa Cruz Biotechnology) and anti-rabbit HRP linked antibodies (CST) were used and detected using the Chemidoc imaging system, image captured with Image Studio Version 3.1 software (BioRad).

6. Biomarker Analysis—

Differences in IC₅₀ values between subgroups were tested using ANOVA. Differences between SLFN11 high and low groups, defined using the bi-modality index (16), were tested using an un-paired t-test. Spearman rank correlation was used to assess the association between IC50 values and RPPA protein markers, The Benjamini & Hochberg (1995) method (17) was used to control false discovery rate (FDR). RPPA generates a quantitative measurement of expression of over 200 total and phosphorylated proteins, these values can be correlated to other variables such as IC50 values, and be compared between biomarker defined groups using standard statistical techniques. All analyses were performed using the R software.

7. Mouse Strains

For the syngeneic mouse model, 6-week-old female athymic nude mice (Envigo) were used. Animals were maintained in accordance with the Institutional Animal Care and Use Committee (IACUC) of The University of Texas MD Anderson Cancer Center and the NIH Guidelines for the Care and Use of Laboratory Animals. Experiments were performed under protocol: 00001191-RN03.

8. Establishment of Flank Xenografts and Efficacy Studies in Nude Mice

High SLFN11 protein expressing SCLC cell line DMS79 and low SLFN11 protein expressing cell line H865 were used for generation of xenografts. For subcutaneous injections, 1×10⁶ SCLC cells were injected into one flank of each mouse with PBS: Matrigel (1:1, BD Biosciences). At an average tumor volume of 100 mm³, mice were randomly allocated to three groups (n=5). Mice received either Vehicle (0.9% saline), lurbinectedin (0.1 mg/kg i.v. once per week), or cisplatin (6 mg/kg i.p. once per week). Tumor volumes were measured every other day using digital calipers.

9. Statistical Analysis

Differences in IC50 values between subgroups were tested using ANOVA. Differences between SLFN11 high and low groups, defined using the bi-modality index were tested using an un-paired t-test. Spearman rank correlation was used to assess the association between IC50 values and RPPA protein markers, The Benjamini & Hochberg (1995) method was used to control false discovery rate (FDR). Differences in vivo drug sensitivity were tested by comparing tumor control ratios using an un-paired t-test.

B. Results.

1. Human SCLC Cells Show Sensitivity to Lurbinectedin Both In Vitro and In Vivo.

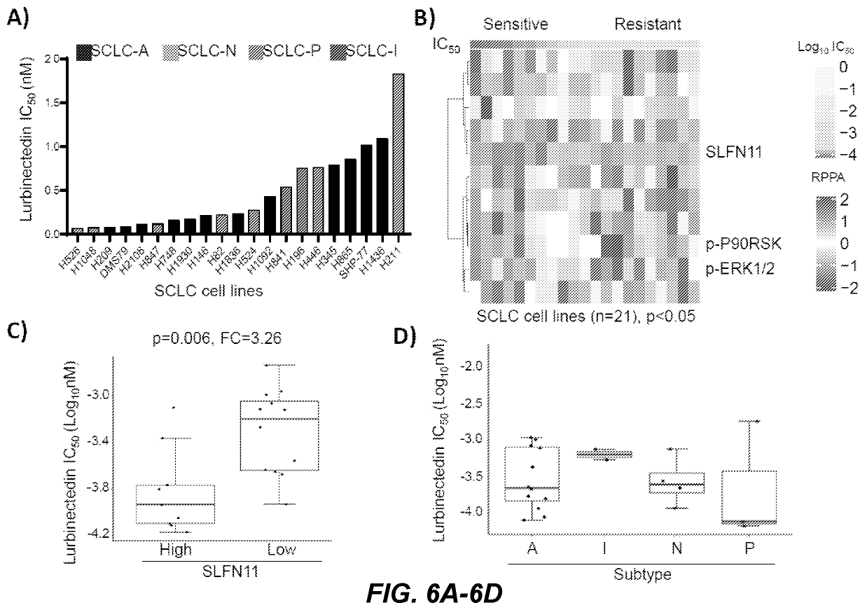

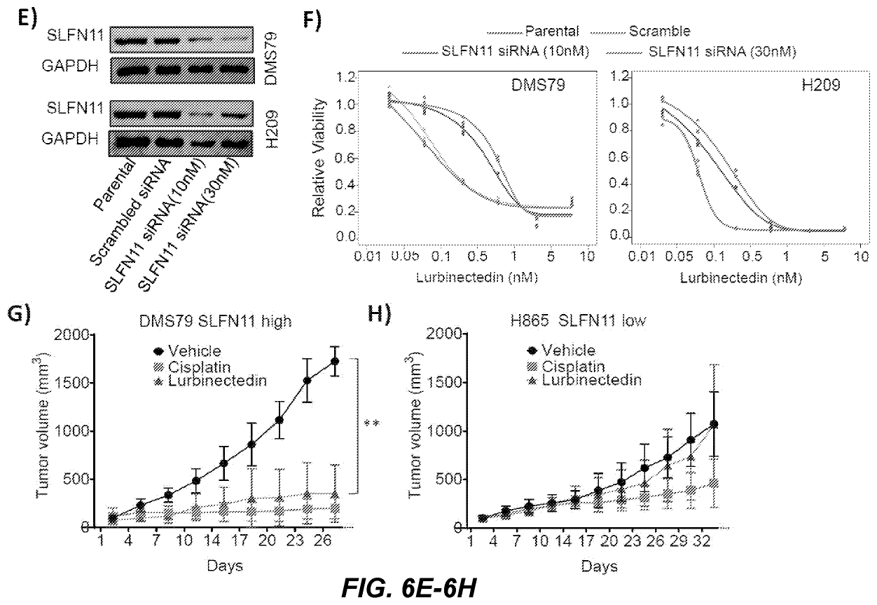

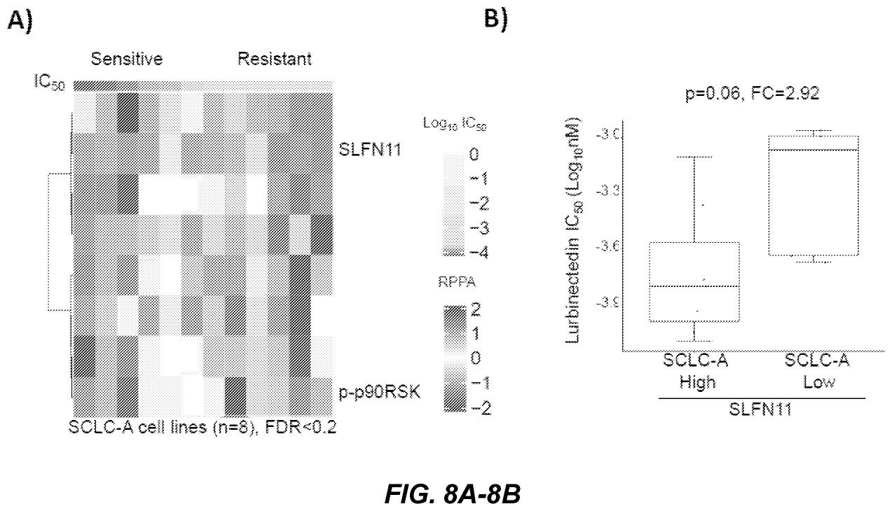

To assess the sensitivity of SCLC to lurbinectedin the inventors screened a panel of 21 human SCLC cell lines, representing all the four SCLC subtypes (A, N, P and I) in cell proliferation assays. Though majority of SCLC cell lines were sensitive to lurbinectedin at a nanomolar range, they showed a range of sensitivities (FIG. 6A). Notably, all IC₅₀ values are lower than the peak plasma concentration of lurbinectedin of 148.2-153.8 ng/mL (188.8-196.0 nM) reported in clinical testing (5). Next, they sought to identify potential protein biomarkers of response leveraging baseline reverse phase protein array (RPPA) proteomic profiling data of the panel of cell lines, which quantifies expression of 209 total and phosphorylated proteins. To identify novel biomarkers of response they correlated IC₅₀ values with the RPPA expression dataset—SLFN1I was identified as the strongest biomarkers of sensitivity to lurbinectedin (R=−0.618, p=0.003), with phosphorylated P90RSK, phosphorylated ERK1/2 and phosphorylated PI3K amongst the top biomarkers of resistance (R=0.664, p=0.001, R=0.474, p=0.030, and R=0.433, p=0.049, respectively) (FIG. 6B, Table S1). Additional activated/mTOR pathway (p-mTOR) was associated with relative resistance to lurbinectedin, but failed to reach statistical significance (p=0.67). As SLFN11 is a known biomarker of sensitivity to chemotherapy and other DNA damaging agents, the inventors performed a secondary analysis dichotomizing the cell lines based on SLFN1I expression which is bimodal in SCLC (10). As shown in FIG. 6C, cell lines with higher SLFN11 expression (bimodal separation by RPPA) were significantly more sensitive to lurbinectedin (p=0.006, FC=3.26). The inventors then compared lurbinectedin cytotoxicity based on a recently published molecularly-defined subtypes of SCLC (SCLC-A, SCLC-N, SCLC-P, SCLC-I), there was, however, no significant difference in the in vitro activity of lurbinectedin between the subtypes (FIG. 6D), a not unsurprising observation as all four subtypes contain models with high SLFN11 expression. To validate SLFN11 as a biomarker of response to lurbinectedin, the inventors first knocked down SLFN11 expression using siRNA in high SLFN11 protein expressing cell lines-DMS79 and 11209 (FIG. 6E) and performed proliferation assays. In both DMS79 and H209, SLFN11 knockdown reduced sensitivity to lurbinectedin as compared to their respective parental and scrambled siRNA controls (FIG. 6F). Second, the inventors compared the in vivo efficacy of lurbinectedin in models representing SLFN11-high and -low disease. In DMS79 xenografts that have high SLFN11 expression, lurbinectedin treatment significantly reduced tumor growth as compared to vehicle (FIG. 6G). However, H865 xenografts, with lower SLFN11 expression, were significantly less sensitive to lurbinectedin, supporting SLFN11 as a biomarker of response to lurbinectedin (FIG. 6H). Since SCLC-A is the most prevalent SCLC subtype (18) and showed a range of in vitro sensitivities to lurbinectedin (FIG. 6D), the inventors repeated the biomarker analysis in SCLC-A cell lines. Again, SLFN11 was identified as a top biomarker of sensitivity, and phosphorylated P90RSK as biomarker of resistance (FIG. 8, Table S2). These observations confirm SLFN11 as a biomarker of response to lurbinectedin

2. Lurbinectedin Induces DNA Damage and Replication Stress.

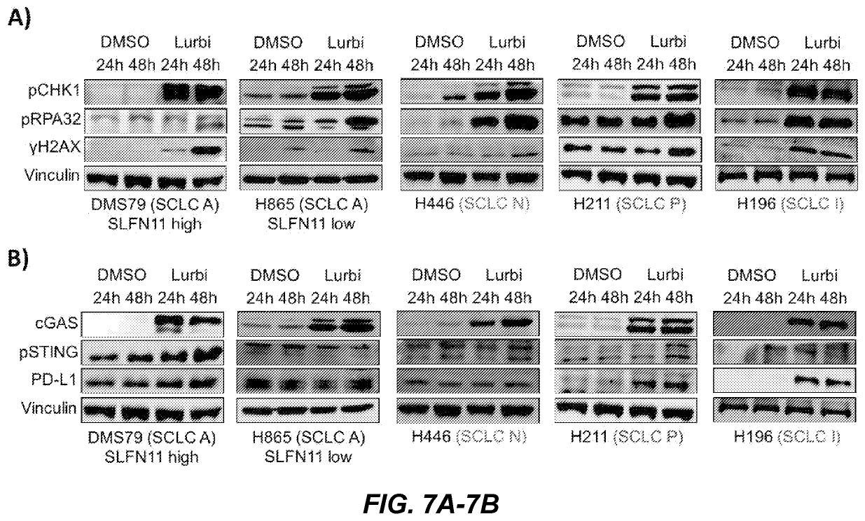

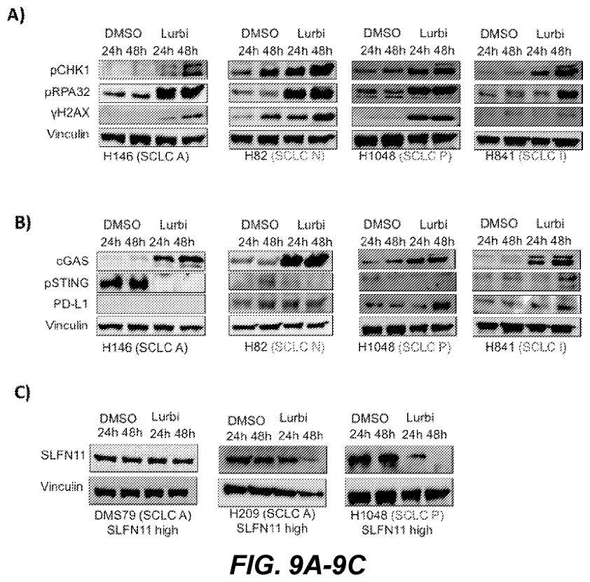

Based on its mechanism of action, the inventors predicted that lurbinectedin would lead to increased replication stress and DNA damage in SCLC models. To test this, SCLC cells from all four subtypes were treated with lurbinectedin (0.9 nM/L) for 24 and 48h and markers of DNA damage and replication stress were assayed by Western blot. All cell lines tested showed increases in markers of replication stress—phospho CHK1(S345) and phospho RPA32(S4/S8)—as compared to DMSO control (FIGS. 7A and 9A). Similar increases in phospho TH2AX were also observed, indicating DNA double-strands breaks in SCLC cells upon treatment with lurbinectedin (FIGS. 7A and 9A).

3. Lurbinectedin Treatment Activates the cGAS-STING Pathway and Increases PD-L1 Expression.

As the inventors' group has shown that DNA damage induces release of cytosolic DNA and increases PD-L1 level via activation of the cGAS-STING pathway in pre-clinical models of SCLC, (21,22) the inventors sought to test if lurbinectedin also induces PD-L1 expression via activation of cGAS-STING signaling. Interestingly, treatment of SCLC cells with lurbinectedin for 24 and 48h induced an increase in protein levels of cGAS in all cell lines tested, as compared to DMSO control (FIGS. 7B and 9C). However, a corresponding increase in phospho STING and PD-L1 was only observed in cell lines from the SCLC-P and SCLC-I subtypes (FIGS. 7B and 9B). These data suggest potential for a synergistic interaction between lurbinectedin and anti-PD-L1 treatment, but that further testing is required to identify potential biomarkers for this interaction to explain the variation seen in the cell lines tested, and to test the combination in immune competent models of SCLC.

As treatment with other DNA damaging agents (cisplatin, PARP inhibitors) can reduce expression of SLFN11, and reports have shown that the activation of the cGAS-IFN axis can induce SLFN11 expression, the inventors were interested in understanding the effect of lurbinectedin on SLFN11. In three cell lines with high SLFN11 expression—DMS-79, H209, H1048—lurbinectedin caused variable changes in SLFN11 levels ranging from a minimal decrease (DMS-79), to reduced, to an undetectable level (H1048) (FIG. 9C). The inventors hypothesize that these variable changes are reflective of a balance between epigenetic down-regulation related to treatment, and up-regulation via activation of the cGAS-IFN pathway—further testing of this relationship, including in immune competent systems, will be required to fully understand this relationship (13).

C. Discussion.

The lack of effective therapies and predictive biomarkers are major challenges in the management of advanced SCLC. For decades, topotecan was the only FDA approved treatment for relapsed SCLC, until the recent accelerated FDA approval of lurbinectedin in the second line setting for metastatic SCLC. In the current study the inventors explored the therapeutic efficacy of lurbinectedin in a large number of proteomically profiled SCLC cell lines as well as in cell line xenograft models as a single-agent. Lurbinectedin showed robust cytotoxic effect in SCLC cell lines with IC₅₀ values ranging from 0.06 nM to 1.83 nM (FIG. 6A), all comfortably below the reported clinical plasma concentration (5).

The data provided herein demonstrate that SLFN11 is a top candidate predictive biomarker of response to single-agent lurbinectedin. SLFN11 IHC could easily be translated into clinical setting and be immediately leveraged in ongoing and future clinical trials studying lurbinectedin treatment strategies for patients with SCLC.

D. Tables

TABLE S1
Correlation of IC50 values to lurbinectedin and
RPPA values across all cell lines.

| RPPA target | Correlation | p-value |
|---|---|---|
| mTOR | 0.685 | <0.001 |
| p-p90RSK (T359) | 0.664 | 0.001 |
| SLFN11 | −0.618 | 0.003 |
| P53 | 0.562 | 0.008 |
| B7.H4 | −0.517 | 0.016 |
| RecQ4 | −0.475 | 0.029 |
| p-ERK1/2 (S217/221) | 0.474 | 0.030 |
| CHK1 | −0.455 | 0.038 |
| WRN | 0.448 | 0.041 |
| cKIT | −0.434 | 0.049 |
| PI3K | 0.434 | 0.049 |

TABLE S2
Correlation of IC50 values to lurbinectedin and
RPPA values in SCLC-A cell lines.

| RPPA target | Correlation | p-value |
|---|---|---|
| p-cJUN (S73) | −0.867 | <0.001 |
| SLFN11 | −0.825 | 0.001 |
| p-STING (S366) | −0.825 | 0.001 |
| B7.H4 | −0.804 | 0.002 |
| mTOR | 0.762 | 0.004 |
| p-p90RSK (T359) | 0.755 | 0.005 |
| P53 | 0.748 | 0.005 |
| ZEB1 | 0.727 | 0.007 |

### E. References

The references cited herein, to the extent that they provide exemplary procedural or other details supplementary to those set forth herein, are specifically incorporated herein by reference.
1. Rudin C M, Brambilla E, Faivre-Finn C, et al. Small-cell lung cancer. Nat Rev Dis Primers 2021; 7:3.
2. Horn L, Mansfield A S, Szczesna A, et al. First-Line Atezolizumab plus Chemotherapy in Extensive-Stage Small-Cell Lung Cancer. N Engl J Med 2018; 379:2220-9.
3. Paz-Ares L, Dvorkin M, Chen Y, et al. Durvalumab plus platinum-etoposide versus platinum-etoposide in first-line treatment of extensive-stage small-cell lung cancer (CASPIAN): a randomised, controlled, open-label, phase 3 trial. Lancet 2019; 394:1929-39.
4. NCCN. http://www.nccn.org/professionals/physician_gls/pdf/sclc.pdf.
5. Trigo J, Subbiah V, Besse B, et al. Lurbinectedin as second-line treatment for patients with small-cell lung cancer: a single-arm, open-label, phase 2 basket trial. Lancet Oncol 2020; 21:645-54.
6. Soares D G, Machado M S, Rocca C J, et al. Trabectedin and its C subunit modified analogue PM01183 attenuate nucleotide excision repair and show activity toward platinum-resistant cells. Mol Cancer Ther 2011; 10:1481-9.
7. Vidal A, Munoz C, Guillen M J, et al. Lurbinectedin (PM01183), a new DNA minor groove binder, inhibits growth of orthotopic primary graft of cisplatin-resistant epithelial ovarian cancer. Clin Cancer Res 2012; 18:5399-411.
8. Santamaria Nunez G, Robles C M, Giraudon C, et al. Lurbinectedin Specifically Triggers the Degradation of Phosphorylated RNA Polymerase II and the Formation of DNA Breaks in Cancer Cells. Mol Cancer Ther 2016; 15:2399-412.
9. Belgiovine C, Bello E, Liguori M, et al. Lurbinectedin reduces tumour-associated macrophages and the inflammatory tumour microenvironment in preclinical models. Br J Cancer 2017; 117:628-38.
10. Allison Stewart C, Tong P, Cardnell R J, et al. Dynamic variations in epithelial-to-mesenchymal transition (EMT), ATM, and SLFN11 govern response to PARP inhibitors and cisplatin in small cell lung cancer. Oncotarget 2017; 8:28575-87.
11. Lok B H, Gardner E E, Schneeberger V E, et al. PARP Inhibitor Activity Correlates with SLFN11 Expression and Demonstrates Synergy with Temozolomide in Small Cell Lung Cancer. Clin Cancer Res 2017; 23:523-35.
12. Pietanza M C, Waqar S N, Krug L M, et al. Randomized, Double-Blind, Phase II Study of Temozolomide in Combination With Either Veliparib or Placebo in Patients With Relapsed-Sensitive or Refractory Small-Cell Lung Cancer. J Clin Oncol 2018; 36:2386-94.
13. Zhang B, Ramkumar K, Cardnell R J, et al. A wake-up call for cancer DNA damage: the role of Schlafen 11 (SLFN11) across multiple cancers. Br J Cancer 2021.
14. Tong P, Coombes K R, Johnson F M, et al. drexplorer: A tool to explore dose-response relationships and drug-drug interactions. Bioinformatics 2015; 31:1692-4.
15. Cardnell R J, Feng Y, Mukherjee S, et al. Activation of the PI3K/mTOR Pathway following PARP Inhibition in Small Cell Lung Cancer. PLoS One 2016; 11:e0152584.
16. Wang J, Wen S, Symmans W F, et al. The bimodality index: a criterion for discovering and ranking bimodal signatures from cancer gene expression profiling data. Cancer Inform 2009; 7:199-216.
17. Benjamini Y, Hochberg Y. Controlling the false discovery rate: a practical and powerful approach to multiple testing. JRSS (B) 1995; 57:289-300.
18. Gay C M, Stewart C A, Park E M, et al. Patterns of transcription factor programs and immune pathway activation define four major subtypes of SCLC with distinct therapeutic vulnerabilities. Cancer Cell 2021; 39:346-60 e7.
19. Murai J, Thomas A, Miettinen M, et al. Schlafen 11 (SLFN11), a restriction factor for replicative stress induced by DNA-targeting anti-cancer therapies. Pharmacol Ther 2019; 201:94-102.
20. Cardnell R J, Feng Y, Diao L, et al. Proteomic markers of DNA repair and PI3K pathway activation predict response to the PARP inhibitor BMN 673 in small cell lung cancer. Clin Cancer Res 2013; 19:6322-8.
21. Sen T, Della Corte C M, Milutinovic S, et al. Combination Treatment of the Oral CHK1 Inhibitor, SRA737, and Low-Dose Gemcitabine Enhances the Effect of Programmed Death Ligand 1 Blockade by Modulating the Immune Microenvironment in SCLC. J Thorac Oncol 2019; 14:2152-63.
22. Sen T, Rodriguez B L, Chen L, et al. Targeting DNA Damage Response Promotes Antitumor Immunity through STING-Mediated T-cell Activation in Small Cell Lung Cancer. Cancer Discov 2019; 9:646-61.
23. Jo U, Murai Y, Chakka S, et al. SLFN11 promotes CDT1 degradation by CUL4 in response to replicative DNA damage, while its absence leads to synthetic lethality with ATR/CHK1 inhibitors. Proc Natl Acad Sci USA 2021; 118.
24. Murai J, Feng Y, Yu G K, et al. Resistance to PARP inhibitors by SLFN11 inactivation can be overcome by ATR inhibition. Oncotarget 2016; 7:76534-50.
25. Murai J, Tang S W, Leo E, et al. SLFN11 Blocks Stressed Replication Forks Independently of ATR. Mol Cell 2018; 69:371-84 e6.
26. Murai J, Pommier Y. PARP Trapping Beyond Homologous Recombination and Platinum Sensitivity in Cancers. Annual Review of Cancer Biology 2019; 3:131-50.
27. Byers L A, Bentsion D, Gans S, et al. Veliparib in Combination with Carboplatin and Etoposide in Patients with Treatment-Naive Extensive-Stage Small Cell Lung Cancer: A Phase 2 Randomized Study. Clin Cancer Res 2021.
28. Kang M H, Wang J, Makena M R, et al. Activity of MM-398, nanoliposomal irinotecan (nal-IRI), in Ewing's family tumor xenografts is associated with high exposure of tumor to drug and high SLFN11 expression. Clin Cancer Res 2015; 21:1139-50.
29. Nogales V, Reinhold W C, Varma S, et al. Epigenetic inactivation of the putative DNA/RNA helicase SLFN11 in human cancer confers resistance to platinum drugs. Oncotarget 2016; 7:3084-97.
30. Takashima T, Sakamoto N, Murai J, et al. Immunohistochemical analysis of SLFN11 expression uncovers potential non-responders to DNA-damaging agents overlooked by tissue RNA-seq. Virchows Arch 2021; 478:569-79.
31. Zoppoli G, Regairaz M, Leo E, et al. Putative DNA/RNA helicase Schlafen-11 (SLFN11) sensitizes cancer cells to DNA-damaging agents. Proc Natl Acad Sci USA 2012; 109:15030-5.
32. Gardner E E, Lok B H, Schneeberger V E, et al. Chemosensitive Relapse in Small Cell Lung Cancer Proceeds through an EZH2-SLFN11 Axis. Cancer Cell 2017; 31:286-99.
33. Lheureux S, Oaknin A, Garg S, et al. EVOLVE: A Multicenter Open-Label Single-Arm Clinical and Translational Phase II Trial of Cediranib Plus Olaparib for Ovarian Cancer after PARP Inhibition Progression. Clin Cancer Res 2020; 26:4206-15.
34. Tumini E, Herrera-Moyano E, San Martin-Alonso M, et al. The Antitumor Drugs Trabectedin and Lurbinectedin Induce Transcription-Dependent Replication Stress and Genome Instability. Mol Cancer Res 2019; 17:773-82.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for treating a subject with small cell lung cancer (SCLC) or with a neuroendocrine cancer, the method comprising administering one or more therapeutics to a subject that has had been evaluated for SLFN11 expression in a biological sample from the subject; wherein the one or more therapeutics comprise lurbinectedin.

2. The method of claim 1, wherein the subject was evaluated for SLFN11 expression by evaluating SLFN11 in an immunohistochemistry assay performed on a biological sample from the subject.

3. The method of claim 1 or 2, wherein the subject was evaluated for SLFN11 expression by detecting binding of the SLFN11 protein to an anti-SLFN11 antibody, wherein the anti-SLFN11 antibody comprises clone D8W1B or the anti-SLFN11 antibody HPA23030.

4. The method of any one of claims 1-3, wherein the biological sample comprises a pleural effusion, liquid biopsy, blood, serum, plasma, biopsy, or tissue sample.

5. The method of claim 4, wherein the biological sample comprises circulating tumor cells.

6. The method of any one of claims 1-5, wherein the biological sample comprises circulating tumor DNA.

7. The method of any one of claims 1-6, wherein the expression level of SLFN11 in the biological sample from the subject has been quantitated.

8. The method of claim 7, wherein the expression level of SLFN11 is normalized.

9. The method of any one of claims 1-8, wherein the subject has been determined to be positive for SLFN11 expression in the biological sample.

10. The method of claim 9, wherein the subject has been determined to have SLFN11+cells in the biological sample from the subject.

11. The method of any one of claims 1-10, wherein the subject has or has been determined to have a high level of SLFN11 expression in the biological sample from the subject.

12. The method of claim 11, wherein the subject has or has been determined to have a high level of SLFN11 expression in the biological sample from the subject compared to a control level of expression.

13. The method of claim 12, wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04.

14. The method of any one of claims 1-14, wherein the subject has been determined to have a H-score for SLFN11 of 1 or greater than 1.

15. The method of claim 14, wherein the subject has been determined to have a H-score for SLFN11 of greater than 1.

16. The method of any one of claims 10-15, wherein the therapeutic excludes an ATR inhibitor.

17. The method of claim 16, wherein the therapeutic excludes berzosertib.

18. The method of any one of claims 1-17, wherein the subject has been previously treated with a cancer therapeutic.

19. The method of any one of claims 1-18, wherein the subject has been previously treated with a platinum-based chemotherapeutic.

20. The method of any one of claims 1-17, wherein the subject has not been previously treated with a platinum-based chemotherapy.

21. The method of claim 19 or 20, wherein the platinum-based chemotherapeutic comprises cisplatin, oxaliplatin, and/or carboplatin.

22. The method of any one of claims 1-21, wherein the subject has not previously been treated with lurbinectedin.

23. The method of any one of claims 1-22, wherein the cancer is further defined as recurrent.

24. The method of any one of claims 1-23, wherein the cancer comprises SCLC type A.

25. The method of any one of claim 1-24, wherein the subject is a human subject.

26. A method for evaluating a subject comprising detecting SLFN11 in a biological sample from the subject.

27. The method of claim 26, wherein SLFN11 is detected by immunohistochemistry of a biological sample from the subject.

28. The method of claim 26 or 27, wherein SLFN11 is detected by detecting binding of the SLFN11 protein to an anti-SLFN11 antibody, wherein the anti-SLFN11 antibody comprises clone or the anti-SLFN11 antibody HPA23030.

29. The method of any one of claims 26-28, wherein the subject has SCLC or a neuroendocrine cancer.

30. The method of any one of claims 26-29, wherein the biological sample comprises a liquid biopsy, blood, serum, plasma, biopsy, pleural effusion, or tissue sample.

31. The method of claim 30, wherein the biological sample comprises circulating tumor cells.

32. The method of any one of claims 27-31, wherein the biological sample comprises circulating tumor DNA.

33. The method of any one of claims 26-32, wherein the method comprises or further comprises quantitating the expression level of SLFN11 in the biological sample from the subject.

34. The method of claim 33, wherein the method comprises or further comprises normalizing the expression level of SLFN11.

35. The method of any one of claims 33-34, wherein the method comprises or further comprises comparing the expression level of SLFN11 to a control.

36. The method of any one of claims 26-35, wherein the subject has been determined to be positive for SLFN11 expression in the biological sample.

37. The method of claim 36, wherein the subject has been determined to have SLFN11+cells in the biological sample from the subject.

38. The method of any one of claims 26-37, wherein the subject has or has been determined to have a high level of SLFN11 expression in the biological sample from the subject.

39. The method of claim 38, wherein the subject has or has been determined to have a high level of SLFN11 expression in the biological sample from the subject compared to a control level of expression.

40. The method of any one of claims 35-39, wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04.

41. The method of any one of claims 26-40, wherein the subject has not previously been treated with lurbinectedin.

42. The method of any one of claims 26-41, wherein the subject has been previously treated with a cancer therapeutic.

43. The method of any one of claims 26-42, wherein the subject has been previously treated with a platinum-based chemotherapeutic.

44. The method of any one of claims 26-41, wherein the subject has not been previously treated with a platinum-based chemotherapy.

45. The method of claim 43 or 44, wherein the platinum-based chemotherapeutic comprises cisplatin, oxaliplatin, and/or carboplatin.

46. The method of any one of claims 29-41, wherein the cancer is further defined as recurrent.

47. The method of any one of claims 29-46, wherein the cancer comprises SCLC type A.

48. The method of any one of claim 26-47, wherein the subject is a human subject.

49. The method of any one of claims 26-48, wherein the method further comprises determining the H-score for the level of expression of SLFN11 in the biological sample from the subject.

50. A method for predicting a response to lurbinectedin in a subject having a neuroendocrine cancer or SCLC comprising
a) evaluating SLFN11 in a biological sample from the subject;
b) predicting that the subject will respond to lurbinectedin after (i) SLFN11 expression is detected in the biological sample from the patient; (ii) the patient is determined to have high expression of SLFN11 compared to a control wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04; or (iii) the H-score for the level of expression in the biological sample from the subject is 1 or is greater than 1 or
c) predicting that the subject will not respond to lurbinectedin after (i) SLFN11 expression is not detected in the biological sample from the patient; (ii) the patient is determined to have low or substantially the same level of SLFN11 expression compared to a control wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04; or (iii) iii) the H-score for the level of expression in the biological sample from the subject is less than 1.

51. The method of claim 50, wherein the subject was evaluated for SLFN11 expression by evaluating SLFN11 in an immunohistochemistry assay performed on a biological sample from the subject.

52. The method of claim 50 or 51, wherein the subject was evaluated for SLFN11 expression by detecting binding of the SLFN11 protein to an anti-SLFN11 antibody, wherein the anti-SLFN11 antibody comprises clone D8W1B or the anti-SLFN11 antibody HPA23030.

53. The method of any one of claims 50-52, wherein the biological sample comprises a liquid biopsy, blood, serum, plasma, biopsy, pleural effusion, or tissue sample.

54. The method of claim 53, wherein the biological sample comprises circulating tumor cells.

55. The method of any one of claims 50-54, wherein the biological sample comprises circulating tumor DNA.

56. The method of any one of claims 50-55, wherein the expression level of SLFN11 in the biological sample from the subject has been quantitated.

57. The method of claim 56, wherein the expression level of SLFN11 is normalized.

58. The method of any one of claims 50-57, wherein the subject has been determined to be positive for SLFN11 expression in the biological sample.

59. The method of any one of claims 50-58, wherein the method further comprises treating the subject.

60. The method of claim 59, wherein the subject is predicted to respond to lurbinectedin and the method further comprises treating the subject with one or more therapeutics, wherein the one or more therapeutics comprises lurbinectedin.

61. The method of claim 60, wherein the one or more therapeutic excludes an ATR inhibitor.

62. The method of claim 61, wherein the ATR inhibitor comprises berzosertib.

63. The method of any one of claims 50-61, wherein the subject has not previously been treated with lurbinectedin.

64. The method of any one of claims 50-63, wherein the subject has been previously treated with a cancer therapeutic.

65. The method of any one of claims 50-64, wherein the subject has been previously treated with a platinum-based chemotherapeutic.

66. The method of any one of claims 50-63, wherein the subject has not been previously treated with a platinum-based chemotherapy.

67. The method of claim 65 or 66, wherein the platinum-based chemotherapeutic comprises cisplatin, oxaliplatin, and/or carboplatin.

68. The method of any one of claims 50-63, wherein the cancer is further defined as recurrent.

69. The method of any one of claims 50-68, wherein the cancer comprises SCLC type A.

70. The method of any one of claim 50-69, wherein the subject is a human subject.
